(19) United States
(12) Patent Application Publication
(10) Pub. No.: US 2018/0263909 A1
(43) Pub. Date: Sep. 20, 2018

(54) DUAL LOADED LIPOSOMAL NANOPARTICLES

(71) Applicant: University Of Notre Dame du Lac, South Bend, IN (US)

(72) Inventors: Zihni Basar BILGICER, South Bend, IN (US); Tanyel KIZILTEPE BILGICER, South Bend, IN (US); Jonathan Darryl ASHLEY, South Bend, IN (US)

(73) Assignee: University Of Notre Dame du Lac, South Bend, IN (US)

(21) Appl. No.: 15/760,076

(22) PCT Filed: Sep. 15, 2016
(86) PCT No.: PCT/US16/51986

§ 371 Date: Mar. 14, 2018

Related U.S. Application Data
(60) Provisional application No. 62/218,851, filed on Sep. 15, 2015.

(51) Int. Cl. A61K 9/127; A61K 38/08; A61K 31/65; A61K 47/24; A61K 47/10; A61P 35/00

(52) U.S. Cl. CPC A61K 9/1271; A61K 38/08; A61P 35/00; A61K 47/24; A61K 47/10; A61K 31/65

(57) ABSTRACT

The disclosure provides pharmaceutical compositions and method of using the compositions, wherein the compositions comprise liposomes that contain two or more anticancer drugs. In various embodiments the components of the liposomes can include a) a phospholipid, b) a pegylated lipid, c) an aqueous core, and d) at least one covalently-linked drug-conjugated lipid, an encapsulated drug, or a combination thereof, wherein the drug of the lipid-drug conjugate, encapsulated drug, or both, are anticancer drugs.

### RELATED APPLICATIONS

This application claims priority under 35 U.S.C. § 119(e) to U.S. Provisional Patent Application No. 62/218,851, filed Sep. 15, 2015, which is incorporated herein by reference.

### GOVERNMENT SUPPORT

This invention was made with government support under Grant No. W81XWH-15-1-00177 awarded by the Department of Defense. The government has certain rights in the invention.

### BACKGROUND OF THE INVENTION

Multiple myeloma (MM) is a rare blood cancer for which no cure currently exists. MM is characterized by an overgrowth of plasma cells found in the bone marrow that result in a tumor termed a plasmacytoma. The formation of numerous plasmacytomas is considered MM. Symptoms include anemia, bone pain, and neurological alterations. MM deaths represent 2% of all cancer related deaths.

Treatment options for MM include surgery, radiation, and chemotherapy. Currently available chemotherapeutics aim to target cancerous cells leading to tumor growth suppression, however, several limitations remain. Limitations include cell toxicity, poor biodistribution, and lack of selectivity to the target tumor site.

Due to the common occurrence of drug resistance associated with MM, many patients undergo combination therapy following a relapse. Combination therapy in the oncology field more aggressively combats tumor growth via the disruption of multiple cellular mechanisms that cancerous cells utilize for rapid growth. While the current methods of combination therapy may seem advantageous, there are a number of drawbacks. Although multiple drugs may be administered simultaneously, there is no guarantee they will be delivered to or maintained at the tumor site. Additionally, variations in pharmacokinetic properties, metabolism, and non-uniform biodistribution are frequently observed.

Accordingly, there is a need for new therapies for the treatment of multiple myeloma. There is also a need for drug delivery systems that can target cancer cells and simultaneously deliver two or more therapeutic agents to a tumor site, or to tissue that comprises cancer cells.

### SUMMARY

The invention provides a liposomal nanoparticle drug delivery system comprising two or more anti-cancer therapeutics. The therapeutics can be present in a synergistic molar ratio and can be used in methods of treating cancer. The nanoparticles can be used to encapsulate anti-cancer drugs and pro-drug conjugates including proteasome inhibitors and histone deacetylase inhibitors. Liposomes of the invention can range in size from about 10 nm to about 200 nm. In one embodiment the liposome comprises: a) one or more phospholipids, e.g., a single type of phospholipid, or more than one different types of phospholipids; b) a pegylated lipid, which can form a layer surrounding the bilayer of the liposome; and c) one or more drug-conjugated lipids, encapsulated drugs, or a combination thereof.

The invention therefore provides a liposome comprising:

a) a phospholipid;

b) a pegylated lipid;

c) an aqueous core; and

d) at least two different drug components, wherein the drug components comprise a covalently-linked drug-conjugated lipid, an encapsulated drug, or a combination thereof, and wherein the drug components are anticancer drugs;

wherein the diameter of the liposome is about 5 nm to about 200 nm.

The liposome can include two different drug components, wherein the two different drug components are i) a proteasome inhibitor and an anthracycline, ii) a proteasome inhibitor and a histone deacetylase inhibitor (HDAC inhibitor), or iii) an anthracycline and an HDAC inhibitor. The two different drug components are present in a synergistic ratio, such as a ratio described herein.

The proteasome inhibitor can be, for example, carfilzomib or bortezomib. The anthracycline can be, for example, doxorubicin, amrubicin, daunorubicin, epirubicin, idarubicin, nemorubicin, pixantrone, sabarubicin, or valrubicin. The HDAC inhibitor can be, for example, rocilinostat (ACY-1215) or vorinostat. In other embodiments, the proteasome inhibitor, anthracycline, and/or HDAC inhibitor can be exchanged for other proteasome inhibitors, anthracyclines, and/or HDAC inhibitors described herein.

In one embodiment, a drug-conjugated lipid is present and is selected from the group consisting of a bortezomib-prodrug, a histone deacetylase inhibitor-prodrug, and a doxorubicin-prodrug.

In one embodiment, the phospholipid comprises 1,2-distearoyl-sn-glycero-3-phosphocholine (DSPC), 1,2-distearoyl-sn-glycero-3-phosphatidylserine (DSPS), 1,2-distearoyl-sn-glycero-3-phosphothethanolamine (DSPE), or hydrogenated soy L-α-phosphatidylcholine (HSPC). In one specific embodiment, the phospholipid is DSPC.

In one embodiment, the pegylated lipid comprises mPEG-DSPE where m is about 10 to about 5000, such as DSPE-PEG2000.

In one embodiment, the liposome comprises at least one encapsulated drug component and the encapsulated drug component is localized to the aqueous core. The encapsulated drug can also be located in the lipid bilayer of the liposome. Other drugs and various drug conjugates may also be located in the lipid bilayer and/or they can be localized, at least in part, to the aqueous core. In various embodiment, the at least one encapsulated drug component is doxorubicin, optionally doxorubicin conjugated to a lipid.

In one embodiment, the drug component comprises carfilzomib and a doxorubicin-prodrug, present in a ratio of about 1:1 to about 2:1. In another embodiment, the drug component comprises carfilzomib and doxorubicin, present in a ratio of about 1:1 to about 2:1. In yet another embodiment, the drug component comprises carfilzomib and rocilinostat, present in a ratio of about 1:1 to about 1:25.

The liposomes can have diameters of about 5 nm to about 30 nm, about 30 nm to about 150 nm, about 100 nm to about 130 nm, or about 110 nm to about 120 nm.

The invention also provides a dual-drug loaded liposome comprising:

a) about 85 wt. % to about 95 wt. % 1,2-distearoyl-sn-glycero-3-phosphocholine (DSPC);

b) about 2 wt. % to about 8 wt. % DSPE-PEG2000;

c) an aqueous core comprising 0.5 wt. % to about 3 wt. % of a first drug component; and

d) 0.5 wt. % to about 3 wt. % of a second drug component, wherein the second drug component is different from the first drug component, the first and second drug components are i) a proteasome inhibitor and an anthracycline, ii) a proteasome inhibitor and a histone deacetylase inhibitor (HDAC inhibitor), or iii) an anthracycline and an HDAC inhibitor, wherein the diameter of the liposome is about 100 nm to about 130 nm. The proteasome inhibitor can be carfilzomib and the anthracycline can be doxorubicin conjugated to 1,2-dipalmitoyl-sn-glycero-3-phosphoethanolamine (DPPE) through a hydrazone linkage. The lipid bilayer of the liposome can comprise the DPPE moiety of the doxorubicin conjugated to DPPE.

The invention further provides a method to treat a multiple myeloma in a subject comprising administering to a subject afflicted with cancer, such as multiple myeloma or another cancer described herein, an effective amount of the liposome described herein. The liposome can be administered as a composition for the treatment of multiple myeloma, including refractory multiple myeloma or relapse multiple myeloma. The subject can be a human subject or other mammalian subjects.

The invention additionally provides a method to kill or inhibit the growth of cancer cells comprising contacting the cells with an effective amount of liposomes described herein. The cancer cells can be multiple myeloma or another type of cancer cell described herein.

In one embodiment, the liposomal nanoparticle comprises a proteasome inhibitor, such as carfilzomib, housed within the lipid bilayer, and doxorubicin encapsulated in the aqueous core of the liposome. Carfilzomib and doxorubicin are significantly effective when present in a molar ratio of about 1:1.

In one embodiment, the liposomal nanoparticle comprises a proteasome inhibitor, such as carfilzomib, housed within the lipid bilayer, and a doxorubicin-lipid prodrug chemically linked to the lipid head of a phospholipid of the liposome. Carfilzomib and doxorubicin-lipid prodrug are significantly effective when present in a molar ratio of 1:1.

In one embodiment, the liposomal nanoparticle comprises a proteasome inhibitor, such as carfilzomib, bortezomib or a proteasome inhibitor having a boronic acid moiety such as ixazomib, housed within the lipid bilayer or chemically linked to the lipid head of a phospholipid of the liposome, respectively, and an HDAC inhibitor having a hydroxamic acid moiety such as rocilinostat (ACY-1215) housed in the lipid bilayer. The molar ratio for the proteasome inhibitor and the HDAC inhibitor can be about 2:1 to about 1:1000, typically about 2:1 to about 1:25.

The compositions described herein can be used in a method for treating cancer comprising administration of such a composition to a subject in need of cancer treatment. The compositions can treat cancer by killing or inhibiting the growth of cancer cells via the administration of an effective dose. The disclosure herein further provides novel compositions, preparation methods, and methods of therapeutic use.

The invention thus provides for the use of compositions described herein for the manufacture of medicaments useful for the treatment of cancer in a mammal, such as a human. The medical therapy can be treating cancer, for example, breast cancer, lung cancer, pancreatic cancer, prostate cancer, or colon cancer. The invention also provides for the use of a composition as described herein for the manufacture of a medicament to treat a disease in a mammal, for example, cancer in a human. The medicament can include a pharmaceutically acceptable diluent, excipient, or carrier.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1A-C. Characterization of the synergistic activity of free carfilzomib and free doxorubicin combination treatment at different molar ratios. (A) Chemical structures of carfilzomib and doxorubicin. (B) Combination index (CI) values of the different combinations of free carfilzomib and free doxorubicin were calculated based upon their respective IC₅₀ values to measure the level of synergism (CI<1) or antagonism (CI>1) using the Chou-Talalay method. (C) Cytotoxicity of free carfilzomib, free doxorubicin, and a combination of 1:1 molar ratio at a concentration of 25 nM of each drug against NCIH929 and MM.1S cell lines was determined at 48 h. The combination treatment with 1:1 molar ratio demonstrated synergy in both cell lines. Data represents means of triplicate cultures (±s.d.).

FIG. 2A-C. Synthesis and characterization of carfilzomib and doxorubicin dual drug loaded liposomes. (A) Schematic of the conjugation of doxorubicin to DPPE-GA via a labile hydrazone bond. (B) Illustrations of the single agent loaded liposomes, NP[Dox] (top) and NP[Carf] (middle), and the dual drug loaded liposome, NP[Carf+Dox] (bottom). (C) Representative dynamic light scattering analyses of the different liposomal nanoparticles. NP[Carf], NP[Dox], and NP[Carf+Dox] yielded the same average diameter of ˜115 nm. Data shown is from a representative experiment.

FIG. 3. Release of carfilzomib and doxorubicin from dual drug loaded liposomes. Drug release from NP[Carf+Dox] was performed in phosphate buffered saline (PBS) pH=7.4 over a 72 h period. Data shown is from a representative experiment.

FIG. 4A-D. Cytotoxicity of the dual drug loaded liposomes. (A) Cytotoxicity of free carfilzomib (Carf) and NP[Carf]. (B) Cytotoxicity of free doxorubicin (Dox) and NP[Dox]. (C) Cytotoxicity of Carf+Dox, NP[Carf]+NP[Dox], and NP[Carf+Dox]. All of the cytotoxicity assays were determined at 48 h with NCIH929 cells. (D) Apoptosis in NCI-H929 cells was assessed by flow cytometry following Annexin-V staining. Cells were incubated with the different free and liposomal formulations of carfilzomib and doxorubicin at a concentration of 12.5 nM for each drug. The molar drug ratio of carfilzomib to doxorubicin of 1:1 was used for all combinations. Data represents means of triplicate cultures (±s.d.). *<0.05.

FIG. 5A-B. Determination of maximum tolerated dose of free carfilzomib and free doxorubicin combination treatment in vivo. Tumor bearing SCID mice were injected intravenously on days 1, 2, 8, and 9 with PBS (Control), 1 mg/kg Carf+0.8 mg/kg Dox (1.8 mg/kg Carf+Dox), 1.5 mg/kg Carf+1.2mg/kg Dox (2.7 mg/kg Carf+Dox), or 2 mg/kg Carf+1.6 mg/kg Dox (3.6 mg/kg Carf+Dox). (A) Tumor growth inhibition was measured via calipers. (B) Percent body weight of the animals was used as a measure of systemic toxicity to determine the maximum tolerated dose.

FIG. 6A-D. In vivo efficacy of combination formulations. (A, B) Tumor bearing SCID mice were injected intravenously on days 1, 2, 8, and 9 with PBS, Carf+Dox, or NP[Carf+Dox] with 1 mg/kg carfilzomib+0.8 mg/kg doxorubicin (total drug dose of 1.8 mg/kg). (A) Tumor growth inhibition was measured via calipers. (B) Percent body weight of the animals was used as a measure of systemic toxicity. (C, D) Tumor bearing SCID mice were injected intravenously on days 1, 2, 8, and 9 with PBS, NP[Carf]+NP[Dox], or NP[Carf+Dox]. (C) Tumor growth inhibition was measured via calipers. (D) Percent body weight of the animals was used as a measure of systemic toxicity. *p<0.05.

FIG. 7. Characterization of the synergistic activity of free rocilinostat and free proteasome inhibitor combination treatment at different molar ratios. Combination index (CI) values of the different combinations of free rocilinostat and free carfilzomib (A) or free bortezomib (B) were calculated based upon their respective IC₅₀ values against NCI-H929 cells to measure the level of synergism (CI<1) or antagonism (CI>1) using the Chou-Talalay method.

### DETAILED DESCRIPTION

Embodiments disclosed herein provide nanoparticle-based drug delivery systems and methods for using the systems. In some embodiments, a drug delivery system may include a liposomal nanoparticle having a therapeutic agent encapsulated within the aqueous core of the nanoparticle, covalently linked to phospholipids heads of a liposomes of the nanoparticle, or embedded within the phospholipid bilayer of liposomes of the nanoparticle.

Combinatorial therapies continue to play a critical role in the treatment of cancers and in multiple myeloma (MM). Formulations that delivery the drugs at their optimal synergistic ratios at the tumor site are critical for harnessing maximum efficacy of combination treatments. While current combination therapies are somewhat effective, controlling the drug ratio at the tumor is extremely difficult due to differences in the pharmacokinetics, biodistribution, and metabolism of each drug. Nanotechnology can overcome these problems by loading the therapeutics into nanoparticles at the optimal ratio to facilitate their controlled release and increase their accumulation in the tumor due to the EPR effect enabled by angiogenic blood vessels. Recent studies have established that angiogenesis plays a critical role in various hematologic malignancies including MM, providing a strong rationale to exploit nanotechnology in managing this disease. Hence, the unique advantages provided by nanotechnology can be used to formulate more effective combination therapies in MM with enhanced synergy at the tumor site with the long term goal of improved patient outcomes.

This disclosure describes (i) identification and incorporation of proteasome inhibitors and anthracyclines into liposomes, (ii) the synthesis of a histone deacetylase (HDAC) inhibitor prodrug, (iii) the identification and incorporation of proteasome inhibitors and HDAC inhibitors into liposomes for improved therapeutic efficacy, (iv) the combination of two or more free drugs from the classes of the above drug classes, and (v) the combination of two or more drugs from the above classes in nanoparticles.

To demonstrate the feasibility of anthracyclines and proteasome inhibitor nanoparticles, different carfilzomib-to-doxorubicin molar ratios (1:100 to 10:1) were first screened against cancer cell lines in order to determine the optimal synergistic ratio (1:1 molar ratio) using the Chou-Talalay method. These nanoparticles demonstrated improved synergy in vitro and were more efficacious in inhibiting tumor growth in vivo than a combination of free drug counterparts or single agent liposomal nanoparticles. This combinatorial nanoparticle formulation ensures that both drugs, e.g., FDA approved drugs, reach the tumor site at their optimal synergistic ratio for maximal anti-cancer efficacy and improved patient outcomes. These methods can be applied to all therapeutic agents within the proteasome inhibitor and anthracycline drug classes.

The drug combinations of proteasome inhibitors and HDAC inhibitors were evaluated for synergy over a wide range of proteasome inhibitor-to-HDAC inhibitor molar ratios (1:5 to 1:1000). Synergy, determined by in vitro screening, was observed for several molar ratios tested. These drug combinations can be incorporated into nanoparticles at their optimal molar ratio for improved synergy in vitro and improved efficacy in inhibiting tumor growth in vivo than a combination of free drug counterparts or single agent liposomal nanoparticles. These combinatorial nanoparticle formulations ensure that the drugs reach the tumor site at their optimal synergistic ratio for maximal anti-cancer efficacy and improved patient outcomes.

While this methodology can be used to create load drugs from any of three types of drug classes into nanoparticles, two different formulations of the dual carfilzomib and doxorubicin loaded nanoparticles are explained in detail. Accordingly, we demonstrate the synthesis and evaluation of NP[Carf+Dox] as an effective means to deliver carfilzomib and doxorubicin to MM tumor cells at their optimal synergistic ratio for improved therapeutic effect. Our results demonstrated that NP[Carf+Dox] had improved efficacy in vitro as well as in vivo compared to the free drug combination highlighting the significance of using nanotechnology as a delivery vehicle for combination drugs in achieving better outcomes in MM. Hence, this study, for the first time, demonstrates the synergy between carfilzomib and doxorubicin and their incorporation into nanoparticles for improved therapeutic effect.

In our design, liposomes were selected due to the advantages they possess over other nanoparticle types. Furthermore, using our novel synthetic method we are able to incorporate the lipids and the drug molecules with stoichiometric precision which enabled the incorporation and delivery of the therapeutics at their optimal ratio. NP[Carf+Dox] exhibited high stability and reproducibility, and efficient drug loading at 1 mol %. Based on literature reports, the drug loading of liposomes can be enhanced to 5-10% mol, depending on lipid packing, particle size, and the particular therapeutics. Ongoing studies are being conducted in our lab to increase the loading of carfilzomib and doxorubicin with even higher efficiency to increase the effectiveness of each and every nanoparticle reaching the tumor site.

By combining the enhanced drug delivery capabilities of nanoparticles with successful combination therapies, significant advances in medicine can be made that will have a profound positive impact in the clinic. Our study demonstrated for the first time, the synergy between carfilzomib and doxorubicin and their incorporation into nanoparticles for improved therapeutic effect. Taken together, this study demonstrates the therapeutic potential of these first generation carfilzomib and doxorubicin dual drug loaded liposomal nanoparticles, and provides the preclinical rationale for clinical development and evaluation of NP[Carf+Dox] for improved patient outcomes in MM.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

Various operations may be described as multiple discrete operations in a manner that may be helpful in understanding embodiments. However, the order of description should not be construed to imply that these operations are order dependent.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five, or one to four, for example if the phenyl ring is disubstituted.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The term “about” can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, composition, or embodiment. The term about can also modify the end-points of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths.

As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

The terms “treating”, “treat” and “treatment” include (i) inhibiting a disease, pathologic or medical condition or arresting its development; (ii) relieving the disease, pathologic or medical condition; and/or (iii) diminishing symptoms associated with the disease, pathologic or medical condition. Thus, the terms “treat”, “treatment”, and “treating” can include lowering, stopping or reversing the progression or severity of the condition or symptoms being treated. As such, the term “treatment” can include medical and/or therapeutic administration, as appropriate.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

As used herein, a “therapeutic agent” can be any type of molecule used in the treatment, cure, prevention, or diagnosis of a disease or other medical condition. Examples of therapeutic agents include, but are not limited to, drugs (e.g., anticancer drugs) and nucleic acids (e.g., siRNA, DNA). Specific examples of therapeutic agents that can be included in the liposomal nanoparticles described herein include, but are not limited to, bortezomib, carfilzomib, doxorubicin and rocilinosta.

As used herein, the terms “encapsulated drug” and “encapsulated drugs” refer to therapeutic agents localized to the aqueous core of the liposome. In some cases, a drug may also exist as covalently attached to the outer lipid heads due to spontaneous formation of the liposome. The term encapsulated drugs refers to anticancer drugs such as anthracyclines and histone deacetylase inhibitors and their respective prodrug derivatives.

As used herein, the term “drug-conjugated lipid” refers to a therapeutic agent that is covalently linked or conjugated to a lipid head and/or embedded within a phospholipid bilayer. Examples include carfilzomib, bortezomib-prodrug, doxorubicin-lipid prodrug, and histone deacetylase inhibitor prodrug.

Proteasomes are proteins responsible for degradation of misfolded proteins, and they play a role in cell signaling pathways. As a result, proteasome inhibitors are important in the area of oncology. As used herein, the terms “proteasome inhibitor” and “proteasome inhibitors” refer to molecules having a function directed towards blocking proteasomal activity, which is required for the breakdown of proteins. Examples of proteasome inhibitors include, but are not limited to, carfilzomib, bortezomib, and bortezomib-prodrugs.

Anthracyclines are a commonly used class of drugs used for the treatment of cancer. Anthracyclines act to inhibit RNA and DNA synthesis by intercalating between base pairs. Although anthracyclines have been the most widely used drug to treat cancer, cardiotoxicity limits their use due to the associated adverse side effects. Anthracyclines include, but are not limited to, doxorubicin, daunorubicin, and idarubicin. For the purpose of this disclosure, doxorubicin-prodrug is considered an anthracycline.

Histone deacetylases (HDACs) are a class of enzymes that remove acetyl groups from an \grave{ε}-N-acetyl lysine amino acid on a histone, which allows the histone to wrap more tightly around DNA. Histone deacetylase inhibitors act to block the coiling process of histone deacetylase thereby inhibiting DNA replication. HDAC inhibitors include, but are not limited to, vorinostat and rocilinostat. Rocilinostat is the generic name for ACY-1215. For the purpose of this disclosure, an HDAC inhibitor-prodrug is considered an HDAC inhibitor.

As used herein, the term bortezomib-prodrug refers to a bortezomib molecule having a reversible boronic ester bond and being conjugated to a lipid. Upon entrance into the body, the bortezomib-prodrug is metabolized into the active molecule bortezomib.

As used herein, the term histone deacetylase inhibitor-prodrug refers to an HDAC inhibitor that has a hydroxamic acid moiety. Upon entrance into the body, the HDAC inhibitor-prodrug is metabolized into the active HDAC inhibitor molecule.

As used herein, the term doxorubicin-prodrug refers to a doxorubicin molecule conjugated to a lipid.

As used herein, the terms “liposome” and “liposomes” refer to a spherical structure having at least one lipid bilayer. A liposome can be used for the administration of therapeutic agents. A liposome can comprise a combination of one or more phospholipids, an optional lipid that is not a phospholipid, such as cholesterol, pegylated lipids, or a combination thereof. As used herein, a liposome may have a diameter of about 30 nm to about 200 nm. In one embodiment, the diameter of the liposomes is about 75 nm to about 125 nm. In certain embodiments, the liposomes can have diameters precisely falling within 110 nm and 125 nm.

As used herein, a liposome may include a micelle having a diameter of about 5 nm to about 29 or 30 nm.

Examples of lipids include 1,2-distearoyl-sn-glycero-3-phosphocholine (DSPC), 1,2-distearoyl-sn-glycero-3-phosphatidylserine (DSPS), 1,2-distearoyl-sn-glycero-3-phosphothethanolamine (DSPE), and Hydro Soy PC, also known as hydrogenated soy L-α-phosphatidylcholine (HSPC), CAS Number 97281-48-6, a versatile phospholipid useful for preparing micelles or liposomes.

An example of a pegylated lipid is 1,2-distearoyl-sn-glycero-3-phosphoethanolamine-N-[carboxy(polyethylene glycol)-2000] (DSPE-PEG2000). Polyethylene glycol (PEG) can be branched having three to ten PEGs chains emanating from a central core group, star PEGs having 10 to 100 PEG chains emanating from a central core group, and comb PEGs having multiple PEG chains grafted onto a polymer backbone.

The term lipid includes mono-, di-, and tri-acylglycerols, phospholipids, free fatty acids, fatty alcohols, cholesterol, cholesterol esters, and the like. Phospholipid refers to a glycerol phosphate with an organic headgroup such as choline, serine, ethanolamine or inositol, having zero, one or two fatty acids esterified to the glycerol backbone.

Multiple myeloma, which may also be referred to as MM, is an extremely rare blood cancer for which there is currently no cure. Multiple Myeloma is characterized by an extreme overgrowth of plasma cells that result in a tumor called a plasmacytoma. There are various types of Multiple Myeloma including refractory Multiple Myeloma and relapse Multiple Myeloma. Refractory Multiple Myeloma as used herein refers to a form of MM in which the patient is unresponsive to treatment. Relapse Multiple Myeloma as used herein refers to a form of MM in which initially the patient was responsive to treatment but is either no longer responsive to treatment or has relapsed.

As used herein, a nanoparticle is defined as a particle having a diameter no greater than 250 nm, typically no greater than about 200 nm. A nanoparticle includes but is not limited to a liposome.

In one embodiment, a liposome can include carfilzomib embedded in the lipid bilayer and doxorubicin in the aqueous core at a 1:1 drug molar ratio. The diameter of the liposome can range from about 10 nm to about 200 nm, typically about 115 nm.

In one embodiment, a liposome can include carfilzomib embedded in the lipid bilayer and doxorubicin-prodrug covalently linked to a phospholipid headgroup at a 1:1 drug molar ratio. The diameter of the liposome can range from about 10 nm to about 200 nm, typically about 115 nm.

In one embodiment, a liposome can include bortezomib-prodrug covalently linked to a phospholipid headgroup with an HDAC inhibitor-prodrug (e.g., ACY-1215) in the aqueous core in an amount effective for inhibiting a disease or symptoms of the disease, including cancer. The diameter of the liposome can range from about 10 nm to about 200 nm, typically about 115 nm.

In one embodiment, a liposome can include carfilzomib embedded within the phospholipid bilayer with rocilinistat prodrug in the aqueous core in an amount effective for inhibiting a disease or symptoms of the disease, including cancer. The diameter of the liposome can range from about 10 nm to about 200 nm, typically about 115 nm.

The disclosure also provides methods for treating cancer in a patient. The methods can include contacting a cancer cell with a pharmaceutical composition described herein. The methods can also include administering to a subject in need of cancer therapy an effective amount of a pharmaceutical composition described herein. The composition (e.g., a composition of nanoparticles described herein) can include a drug-conjugated lipid or an encapsulated drug, or a combination thereof, wherein the drug is effective for treating the cancer, and wherein the composition is taken up by cancer cells, for example, in the subject, and the composition releases the drug to the cancer cells. The cancer cells are thereby killed, or inhibited from growing, thereby treating the cancer.

Useful dosages of the compositions described herein can be determined by comparing their in vitro activity and in vivo activity in animal models. Methods for the extrapolation of effective dosages in mice, and other animals, to humans are known to the art; for example, see U.S. Pat. No. 4,938,949 (Borch et al.). The amount (e.g., mass) of liposomes required for use in treatment will vary not only with the particular active compound of the nanoparticles but also with the route of administration, the nature of the condition being treated, and the age and condition of the patient, and will be ultimately at the discretion of an attendant physician or clinician.

The nanoparticles described herein can be effective anti-tumor compositions and have higher potency and/or reduced toxicity as compared to the corresponding free active drug in the nanoparticles. The invention provides therapeutic methods of treating cancer in a mammal, which involve administering to a mammal having cancer an effective amount of a nanoparticles composition described herein. A mammal includes a primate, human, rodent, canine, feline, bovine, ovine, equine, swine, caprine, bovine and the like. Cancer refers to any various type of malignant neoplasm, for example, colon cancer, breast cancer, melanoma and leukemia, and in general is characterized by an undesirable cellular proliferation, e.g., unregulated growth, lack of differentiation, local tissue invasion, and metastasis.

### Embodiments of the Invention

The invention provides a technique to package two or more therapeutic agents within a single nanoparticle, thus eliminating the batch-to-batch variability associated with the synthesis of liposomal nanoparticles. The PEGylated liposomal nanoparticles described herein have an increased capability for drug loading and control over nanoparticle size compared to previous liposome preparations. PEGylation is the process of covalent and non-covalent attachment or amalgamation of polyethylene glycol (PEG) polymer chains to biomolecules or particles. The addition of pegylated lipids to the liposomal nanoparticle significantly reduces toxicity and the potential for rejection by the host's immune system.

Currently the production of nanoparticles consists of post insertion, which is a process when the liposomal nanoparticle is completely synthesized and the therapeutic agent is forced or inserted into the core of the nanoparticle. This process is flawed in two major ways: first, there is variability in the concentration of the encapsulated therapeutic agent, and second the size of the nanoparticle can vary. As stated earlier, the batch-to-batch variability associated with the production of liposomal nanoparticles is currently a rate-limiting step for a wider market use of such nanoparticle drug delivery systems. Due to the enhanced permeability retention (EPR) effect certain size parameters of the nanoparticle are crucial. EPR is a phenomenon that commonly occurs as a result of cancerous growth. The vascular tissue around the tumor site is much larger than the healthy vascular tissue thereby allowing for the transport of larger molecules such as nanoparticles into the blood thereby reducing the tumor size without affecting the healthy cells.

The therapeutic agents loaded into the pegylated liposomal nanoparticle system include a combination of a proteasome inhibitor and an anthracycline, or a proteasome inhibitor and a histone deacetylase inhibitor (HDAC inhibitor), or any combination of the three classes of drugs. The proteasome inhibitor can be, for example, carfilzomib of bortezomib, the anthracycline can be doxorubicin, amrubicin, daunorubicin, epirubicin, idarubicin, nemorubicin, pixantrone, sabarubicin, or valrubicin, and the HDAC inhibitor can be rocilinostat (ACY-1215), abexinostat (PCI-24781), belinostat (PXD101), chidamide, entinostat (MS-275; SB939), givinostat (ITF2357), kevetrin, mocetinostat (MGCD0103), panobinostat (LBH589), phenylbutyrate, quisinostat (JNJ-26481585), resminostat (4SC-201), romidepsin, trapoxin B, trichostatin A, valproic acid, vorinostat (SAHA), AR-42, CG200745, CHR-2845, CHR-3996, CI994, CUDC-101, HBI-8000, LAQ824, ME-344, or 4SC-202. These drugs and their combinations are currently not available as a dual liposomal nanoparticle formulation. The liposomes described herein can include any two more of these anticancer agents to provide a new form of combination therapy for the treatment of a variety of forms of cancer. The production of such a form of combination therapy for treating cancer, such as MM, drastically increases the efficacy of both drugs and will thus improve the overall quality of life of the patient treated with such compositions.

Depending on the choice of proteasome inhibitor, anthracycline, and HDAC inhibitor, the liposomal nanoparticles can be used to treat cancers such as multiple myeloma, breast cancer, carcinomas such as hepatocellular carcinoma, colon cancer, lung cancer, such as small cell and non-small cell lung cancer, myelomas, such as refractory myelomas, leukemia, such as acute myeloid leukemia, chronic lymphocytic leukemia, and refractory leukemias, lymphoma, such as cutaneous T-cell lymphoma (CTCL), follicular lymphoma, Hodgkin lymphoma, and peripheral T-cell lymphoma (PTCL), ovarian cancer, pancreatic cancer, prostate cancer such as recurrent or metastatic prostate cancer (HRPC), sarcoma, and/or spleen metastasis. The cancer can be, for example, a solid tumor, including solid refractory tumors, or other types of cancer such as a blood-borne cancer (e.g., leukemia).

For example, the dual loaded liposomal nanoparticle can include a proteasome inhibitor, either carfilzomib or bortezomib, loaded within the lipid bilayer. Importantly, a bortezomib prodrug can be produced in order to more effectively encapsulate the drug within the nanoparticle. The anthracycline doxorubicin can either be loaded in the core of the nanoparticle or on the surface of the nanoparticle. The loading of HDAC inhibitor can also be prepared as a prodrug conjugate for similar reasons and can be loaded in the lipid bilayer alongside the selected proteasome inhibitor. The drugs are packaged into the liposomal nanoparticle at specific ratios, in most cases having a ratio of about 1:1. The molar ratio of the drugs is crucial to the effectiveness of the nanoparticles. When the molar ratio is accurate, the synergy observed between the two drugs seemingly becomes a new third drug. In vivo results show that the administration of dual loaded nanoparticles is significantly more effective at reducing the size of tumors associated with MM over the administration of various single loaded nanoparticles.

In summary, the provides a method for preparing dual loaded liposomal nanoparticles at synergistic ratios, thereby enhancing the anticancer efficacy of such nanoparticles. We describe the synthesis and evaluation of dual drug loaded nanoparticles as an effective means to deliver anticancer drugs such as carfilzomib and doxorubicin to multiple myeloma tumor cells at their optimal synergistic ratio. Various molar ratios of carfilzomib to doxorubicin were screened against multiple myeloma cell lines to determine the molar ratio that elicited the greatest synergy using the Chou-Talalay method. The therapeutic agents were then incorporated into liposomes at the optimal synergistic ratio of 1:1 to yield dual drug loaded nanoparticles with a narrow size range of 115 nm and high reproducibility. Our results demonstrated that the dual drug loaded liposomes exhibited synergy in vitro and were more efficacious in inhibiting tumor growth in vivo than a combination of free drugs, while at the same time reducing systemic toxicity. Taken together, this study presents the synthesis and pre-clinical evaluation of dual drug loaded liposomes containing carfilzomib and doxorubicin for enhanced therapeutic efficacy to improve patient outcome in multiple myeloma.

### Pharmaceutical Formulations

The liposomal compositions described herein can be used to prepare therapeutic pharmaceutical compositions, for example, by combining the liposomal compositions with a pharmaceutically acceptable diluent, excipient, or carrier. The liposomal compositions described herein can be formulated as pharmaceutical compositions and administered to a mammalian host, such as a human patient, in a variety of forms. The forms can be specifically adapted to a chosen route of administration, e.g., oral or parenteral administration, by intravenous, intramuscular, topical or subcutaneous routes.

The liposomal compositions described herein may be systemically administered in combination with a pharmaceutically acceptable vehicle, such as an inert diluent or an assimilable edible carrier. For oral administration, liposomal compositions can be enclosed in hard or soft shell gelatin capsules, compressed into tablets, or incorporated directly into the food of a patient's diet. Liposomal compositions may also be combined with one or more excipients and used in the form of ingestible tablets, buccal tablets, troches, capsules, elixirs, suspensions, syrups, wafers, and the like. Such compositions and preparations typically contain at least 0.1% of active liposomal compositions by weight. The percentage of the liposomal compositions and preparations can vary and may conveniently be from about 0.5% to about 60%, about 1% to about 25%, or about 2% to about 10%, of the weight of a given unit dosage form. The amount of active compound in such therapeutically useful compositions can be such that an effective dosage level can be obtained.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid and the like; and a lubricant such as magnesium stearate. A sweetening agent such as sucrose, fructose, lactose or aspartame; or a flavoring agent such as peppermint, oil of wintergreen, or cherry flavoring, may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the liposomal compositions, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the active compound may be incorporated into sustained-release preparations and devices.

The liposomal compositions may be administered intravenously or intraperitoneally by infusion or injection. Formulations of the liposomal compositions can be prepared in water, optionally mixed with a nontoxic surfactant. Dispersions can be prepared in glycerol, liquid polyethylene glycols, triacetin, or mixtures thereof, or in a pharmaceutically acceptable oil. Under ordinary conditions of storage and use, preparations may contain a preservative to prevent the growth of microorganisms.

Pharmaceutical dosage forms suitable for injection or infusion can include sterile aqueous solutions, dispersions, or sterile powders comprising the active ingredient adapted for the extemporaneous preparation of sterile injectable or infusible solutions or dispersions. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained by the maintenance of the required particle size in the case of dispersions, or by the use of surfactants. The prevention of the action of microorganisms can be brought about by various antibacterial and/or antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by agents delaying absorption, for example, aluminum monostearate and/or gelatin.

Sterile injectable dispersions can be prepared by incorporating the liposomal compositions in a required amount in an appropriate solvent or carrier with various other ingredients enumerated above, as required, optionally followed by sterilization. In the case of sterile powders for the preparation of sterile injectable solutions or dispersions, methods of preparation can include vacuum drying and freeze drying techniques, which yield a powder of the liposomal compositions plus any additional desired ingredient present in the solution or dispersion.

Useful dosages of the liposomal compositions described herein can be determined by comparing their in vitro activity, and in vivo activity in animal models. Methods for the extrapolation of effective dosages in mice, and other animals, to humans are known to the art; for example, see U.S. Pat. No. 4,938,949 (Borch et al.). The amount of a compound, or an active salt or derivative thereof, required for use in treatment will vary not only with the particular compound or salt selected but also with the route of administration, the nature of the condition being treated, and the age and condition of the patient, and will be ultimately at the discretion of an attendant physician or clinician.

In general, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day. The compound is conveniently formulated in unit dosage form; for example, containing 1 to 1000 mg, conveniently 5 to 750 mg, most conveniently, 10 to 500 mg of active drug per unit dosage form. In one embodiment, the invention provides a composition comprising a liposomal compositions of the invention formulated in such a unit dosage form.

The liposomal compositions described herein can be conveniently administered in a unit dosage form, for example, containing 5 to 1000 mg/m², conveniently 10 to 750 mg/m², most conveniently, 50 to 500 mg/m² of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by injection or infusion.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Methods and Materials

Polycarbonate membranes (0.1 μm), mini-extruder, methoxy PEG2000-DSPE (mPEG2000) lipids, 1,2-distearoyl-sn-glycero-3-phosphocholine (DSPC), and 1,2-dipalmitoyl-sn-glycero-3-phosphoethanolamine-N-(glutaryl) (DPPE-GA) were from Avanti Polar Lipids, Inc. (Alabaster, Ala.). Carfilzomib was obtained from ChemieTek (Indianapolis, Ind.). All other chemicals were obtained from Sigma-Aldrich (St. Louis, Mo.).

Cell Culture. MM.1S and NCI-H929 cell lines were obtained from American Type Culture Collection (Rockville, Md.) and were cultured according to the vendor's instructions. Both cell lines were cultured in RPMI 1640 media (Cellgro, Manassas, Va.) supplemented with 10% fetal bovine serum (FBS), 2 mM L-glutamine (Gibco, Carlsbad, Calif.), 100 U/mL penicillin, and 100 μg/mL streptomycin (Gibco). NCI-H929 cells were supplemented with an additional 10% FBS and 55 μM 2-mercaptoethanol.

Cytotoxicity and Synergy Analysis. 2×10⁵ cells/well were plated in a 96-well dish 16 h prior to the experiment. Cells were treated with respective cytotoxic agents at varying concentrations. Cytotoxicity was assessed at 48 h using Cell Counting Kit-8 Reagent (Dojindo Molecular Technologies, Rockville, Mass.). Combination index values were calculated using the Chou-Talalay method (Quantitative-analysis of dose-effect relationships—the combined effects of multiple-drugs or enzyme-inhibitors. Adv Enzyme Regul. 1984;22:27-55).

Annexin V Analysis. 2×10⁵ NCI-H929 cells were cultured in the presence of 12.5 nM and 25 nM total drug equivalent concentrations of the different single agent and combination formulations for 24 h. Apoptotic cells were detected with Annexin-V (FITC) antibody (BD Pharmigen, San Diego, Calif.) using a Guava EasyCyte 8HT flow cytometer (EMD Millipore) as previously described (Ashley et al., Liposomal bortezomib nanoparticles via boronic ester prodrug formulation for improved therapeutic efficacy in vivo, J Med Chem. 2014;57(12):5282-5292, which is incorporated herein by reference).

Synthesis of Dox-Lipid. The doxorubicin lipid conjugate was synthesized as previously reported (see Kiziltepe T., Ashley J. D., Stefanick J. F., et al., Rationally engineered nanoparticles target multiple myeloma cells, overcome cell-adhesion-mediated drug resistance, and show enhanced efficacy in vivo, Blood Cancer J. 2012;2:e64, which is incorporated herein by reference). Briefly, 1.14 mL of 25 mg/mL DPPE-GA (34.5 μmol) in chloroform was mixed with 21.4 μL of diisopropylcarbamide (137.9 μmol, 4 eq.) in a 5 mL glass vial and stirred for 5 minutes. Then, 21.4 μL of hydrazine (687 μmol, 20 eq.) was added to the solution, and then stirred for 5 h at room temperature. Solvent was evaporated under vacuum. In a separate vial, 30 mg of doxorubicin (51.7 μmol, 1.5 eq.) was dissolved in 4 mL of methanol and was then added to the vial containing the dried lipid. The solution was stirred for 4.5 days in the dark at room temperature (˜22° C.). Final product was isolated via extraction, and characterized with MALDI-TOF-MS.

Liposome Preparation. Liposomes were prepared by dry film hydration as described previously (see Ashley J. D., Stefanick J. F., Schroeder V. A., et al., Liposomal carfilzomib nanoparticles effectively target multiple myeloma cells and demonstrate enhanced efficacy in vivo. J Controlled Release, 2014;196(0):113-121, which is incorporated herein by reference). Briefly, the lipids, carfilzomib, and dox-lipid were mixed in chloroform, dried to form a thin film, and placed under vacuum overnight to remove residual solvent. The lipid films were hydrated at 65° C. in PBS pH 7.4, gently agitated, and extruded at 65° C. through a 0.1 μm polycarbonate filter. Liposomes prepared adhered to the following molar formula: (95-x-y):5:x:y DSPC:mPEG-DSPE:Carf:Dox-lipid where x and y were either 0 or 1 depending on the desired drug loading of carfilzomib and doxorubicin, respectively.

Particle Sizing. Particle size was observed using dynamic light scattering (DLS) analysis via NanoBrook Omni Particle Size Analyzer (Brookhaven Instruments Corp., Holtsville, N.Y.), using 658 nm light observed at a fixed angle of 90° at 25° C.

Release Analysis of Carfilzomib and Doxorubicin. Liposomes loaded with both carfilzomib and doxorubicin were diluted to 1 mM total lipid concentration. To ensure no free drug was present, the liposome solution was purified via liposome extrusion purification method with a 30 nm polycarbonate membrane as previously described. 100 μL aliquots of the purified liposome solution was placed into each of the 3.5 kDa MWCO Slide-A-Lyzer dialysis units (Thermo Scientific, Waltham, Mass.). Dialysis units were dialyzed together in 1.5 L of PBS at 25° C. 100 μL samples were taken at t=0, 1, 3, 6, 12, 24, 48, and 72 hours for DLS analysis and drug content characterization via RP-HPLC on an Agilent (Santa Clara, Calif.) 1200 series system with a semi-preparative Zorbax C3 column with isopropanol gradients.

MM Xenograft Mouse Model. For all studies, C.B.-17 SCID mice (Charles River Laboratories, Wilmington, Mass.) were irradiated with 150 rad and were inoculated subcutaneously with 5×10⁶NCI-H929 cells. When tumors reached a volume of 50 mm³, mice were randomized into groups and treated intravenously via retro-orbital injections on days 1, 2, 8, and 9. For the combination dosing study, mice were distributed into 4 groups of 3 mice receiving PBS, 1 mg/kg Carf+0.8 mg/kg Dox (1.8 mg/kg), 1.5 mg/kg Carf+1.2 mg/kg Dox (2.7 mg/kg), or 2 mg/kg Carf+1.6 mg/kg Dox (3.6 mg/kg). To compare the NP[Carf+Dox] and Carf+Dox formulations, 3 groups of 8 mice were treated with PBS, Carf+Dox, or NP[Carf+Dox] at a dose of 1.8 mg/kg total drug. For the comparison of the efficacy of the nanoparticle formulations NP[Carf+Dox] and NP[Carf]+NP[Dox], mice were divided into 3 groups of 8 mice and were treated with PBS, NP[Carf]+NP[Dox], or NP[Carf+Dox] at a dose of 2.7 mg/kg carfilzomib and doxorubicin equivalents.

Carfilzomib/NP[Carf] and doxorubicin/NP[Dox] were mixed together prior to each injection for Carf+Dox and NP[Carf]+NP[Dox], respectively. Animals were monitored for body weight and tumor volume. Tumor volume was measured via calipers (volume=0.5×length×(width)). Mice were treated humanely and in accordance with the protocol approved by the Institutional Animal Care and Use Committee (IACUC) at the Freimann Life Science Center (Notre Dame, Ind.).

### Example 1

### Determination of the Optimal Stoichiometric Ratio of Carfilzomib and Doxorubicin for Maximal Synergy

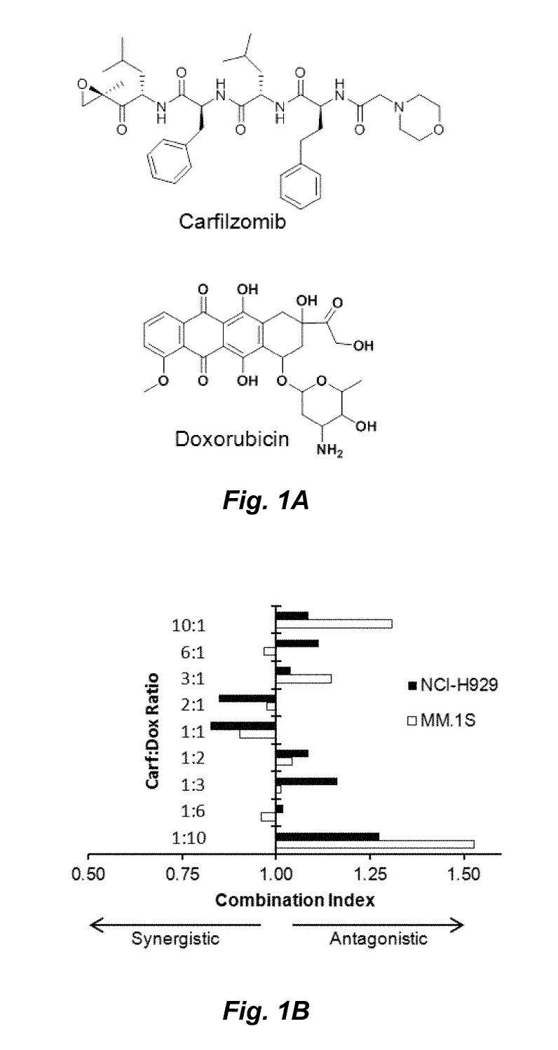

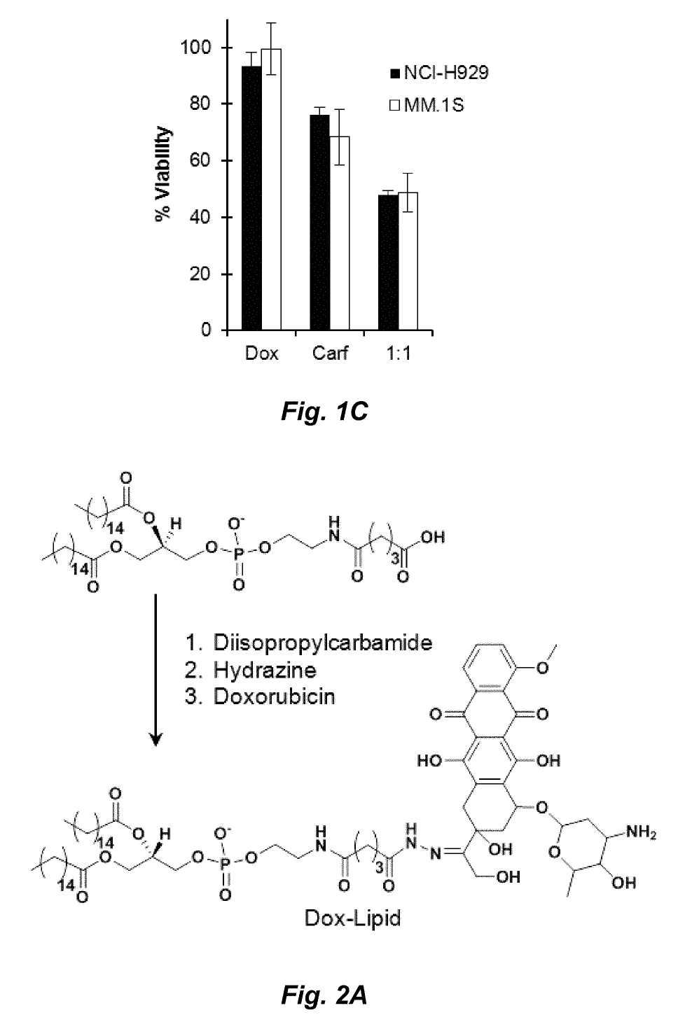

Some anthracyclines and proteasome inhibitors have been found to demonstrate synergy in MM. This provides the rationale for a combination treatment with carfilzomib and doxorubicin for synergistic outcomes (FIG. 1A). Although combination treatments can provide many advantages, the degree of synergism/antagonism between drugs in combination treatments can vary significantly with the drug ratio. Hence, to determine the optimal drug loading of each therapeutic into the nanoparticle, it is essential to first determine the ratio of free drugs that will yield the maximum synergy in vitro. Therefore, we evaluated the cytotoxicity of different molar combinations ranging from 1:10 to 10:1 of free carfilzomib to free doxorubicin using NCI-H929 and MM.1S MM cell lines. The Chou-Talalay method was employed to calculate the combination index (CI) in order to evaluate the synergism (CI<1) or antagonism (CI>1) for each drug combination. Our results demonstrated that the carfilzomib-to-doxorubicin ratios of 1:1 (CI=0.825 for NCI-H929 and 0.903 for MM.1S) and 2:1 (CI=0.847 for NCI-H929 and 0.975 for MM.1S) was effective for synergy of the combination of these two drugs (FIG. 1B). At higher and lower carfilzomib-to-doxorubicin ratios antagonism was observed, highlighting the significance of using optimal ratios for synergistic outcomes. The synergy of the 1:1 ratio is also demonstrated in FIG. 1C, where NCI-H929 and MM.1S cells were incubated with carfilzomib, doxorubicin, or the 1:1 combination at a concentration of 25 nM for each drug. While each drug alone demonstrated only minimal cytotoxicity at 25 nM, the 1:1 combination demonstrated significant synergistic cell death in MM cells. Thus, given the synergy and cytotoxicity observed in MM cell lines, the 1:1 molar drug ratio was selected for nanoparticle formulation.

### Example 2

### Preparation of Carfilzomib and Dox Loaded Liposomal Nanoparticles

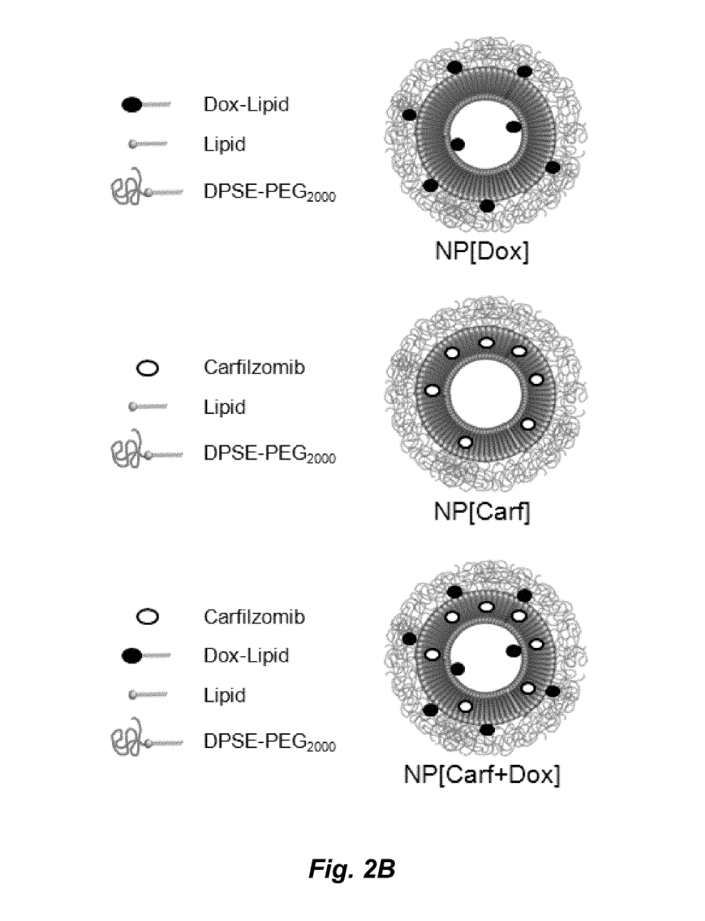

Based on the optimal drug ratio analysis, both therapeutics need to be incorporated into liposomes at a 1:1 molar ratio with controlled drug release so they reach the tumor site at their optimal synergistic ratio for maximum therapeutic efficacy. To incorporate doxorubicin into the nanoparticles, first we conjugated doxorubicin to the polar head group of a DPPE lipid via a hydrolyzable hydrazone bond to create a doxorubicin lipid prodrug conjugate (Dox-lipid; FIG. 2A). The slow hydrolysis of this labile bond facilitates a controlled release of doxorubicin from the nanoparticle surface. The dox-lipid was purified via extraction, mixed with the other lipid constituents at a molar ratio of 94:5:1 DSPC: DSPE-PEG2000 : Dox-lipid to form the lipid film, and then hydrated to form doxorubicin loaded liposomes (NP[Dox]; FIG. 2B). This allowed precise control over the molar ratio of doxorubicin presented on the nanoparticle and ensured high nanoparticle purity and reproducibility.

Carfilzomib can be embedded into the lipid bilayer of liposomes with high efficiency due to its hydrophobicity. Hence, carfilzomib was loaded into liposomes by mixing it with the other lipids prior to film formation at the following molar ratio of 94:5:1 DSPC : DSPE-PEG2000: carfilzomib. This method yielded nanoparticles with high stability, purity, and reproducibility. The drug loading for the carfilzomib loaded liposomes (NP[Carf]) was 1 mol %, equal to NP[Dox] (FIG. 2B).

To make dual drug loaded liposomes (NP[Carf+Dox]), carfilzomib and the dox-lipid were passively loaded into liposomes with high purity and exact stoichiometry. Specifically, carfilzomib and the dox-lipid were mixed with the other lipids at the molar ratio of 93:5:1:1 DSPC : DSPE-PEG2000 : Carfilzomib:Dox-lipid prior to film formation to facilitate their insertion into the bilayer (FIG. 2B). This method ensured that the drugs and lipids were incorporated into the liposomes at precise stoichiometric ratios.

The liposomes were extruded through a 100 nm polycarbonate membrane to yield unilamellar liposomes. NP[Carf+Dox], NP[Carf], and NP[Dox] yielded the same DLS results with an average diameter of 115±1.36 nm with high reproducibility and stability (FIG. 2C). This also was consistent with non-drug loaded liposomes showing that the presence of the therapeutics does not affect the size of the liposomes. Importantly, the diameter of these liposomes falls within the particle size range required for the passive targeting of tumors via the enhanced permeability and retention (EPR) effect.

The loading efficiency for both drugs is important to maintaining the precision of the molar drug ratio and minimizing the variability and impurities during nanoparticle formation. To maintain high loading efficiencies, 1 mol % drug loading into nanoparticles was selected for both carfilzomib and doxorubicin, which yielded loading efficiencies >95% for both drugs. This precluded the requirement for any purification after particle formation to remove any free drug in solution. The synthetic approach used to prepare NP[Carf+Dox] enabled high drug loading efficiencies, narrow size range precision, and homogenous particle populations with minimal batch-to-batch variability.

### Example 3

### Release of Carfilzomib and Doxorubicin from the Dual Drug Loaded Liposomes

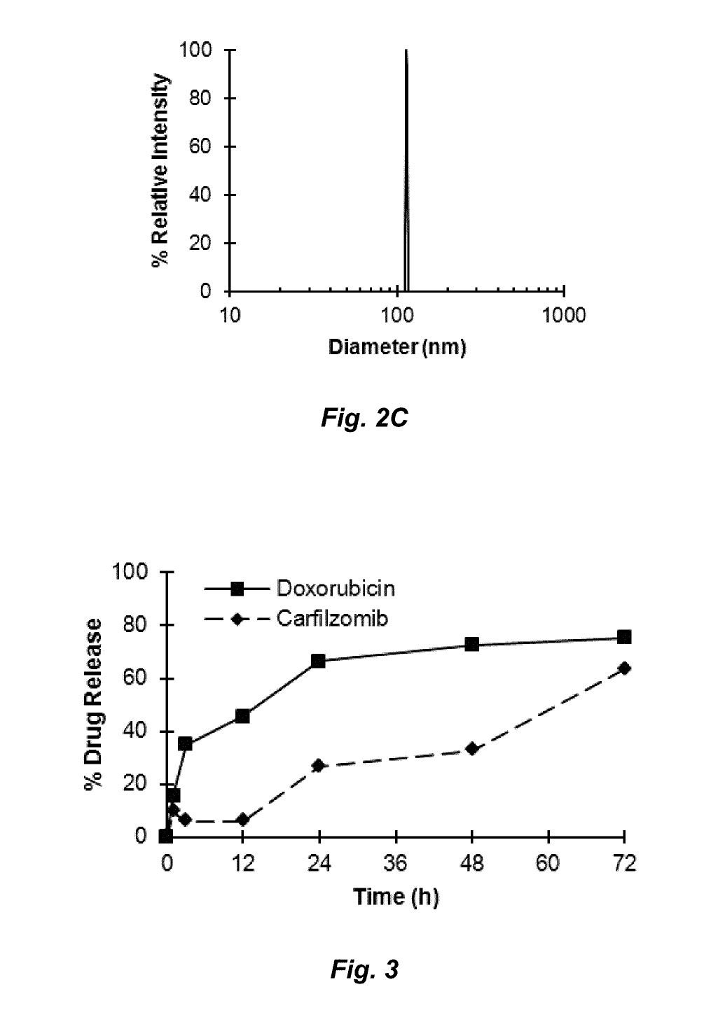

After loading carfilzomib and doxorubicin into liposomes at the optimal synergistic ratio, we evaluated their release from NP[Carf+Dox] using dialysis in conjunction with HPLC analysis. The results showed that both drugs were retained and released slowly from the nanoparticle over a 72 hour period (FIG. 3). Doxorubicin was released more rapidly than carfilzomib from NP[Carf+Dox], which could be attributed to the differences in the mechanism of release for each drug. Doxorubicin, being surface conjugated to the nanoparticle, requires the hydrolysis of the hydrazone bond before being released, whereas carfilzomib is released via diffusion out of the lipid bilayer which enables the slow release of the drug. Although carfilzomib and doxorubicin were released at different rates, the nanoparticles were able to maintain a synergistic drug ratio between 1:1 and 2:1 starting at 24 hours when the nanoparticles maximally accumulate in the tumor. These results demonstrate that NP[Carf+Dox] released both drugs in a controlled manner to facilitate the delivery of therapeutics to the tumor site at their optimal synergistic ratio.

### Example 4

### In Vitro Evaluation of the Dual Drug Loaded Liposomes

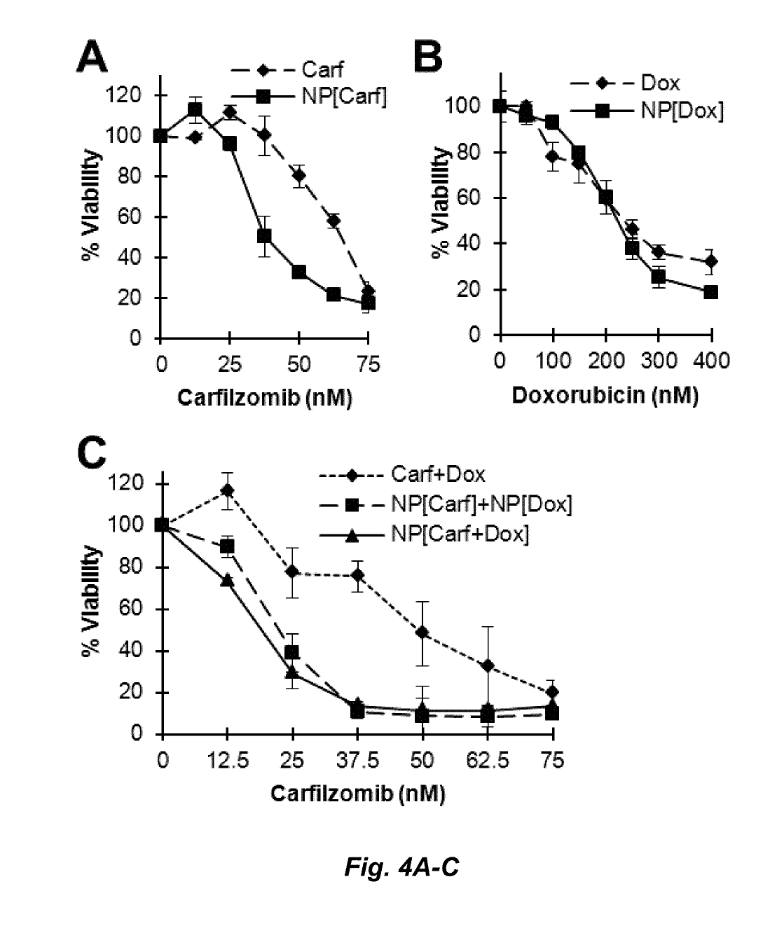

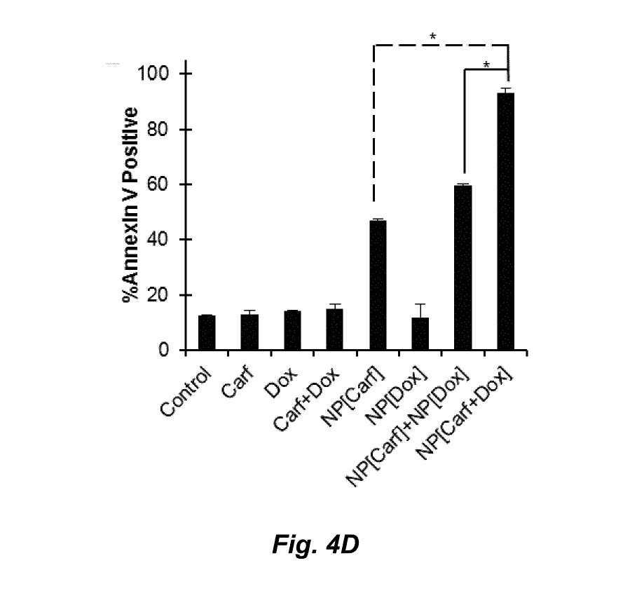

After having engineered NP [Carf+Dox], we evaluated its efficacy on MM cells in vitro. First, the cytotoxicity of the free drugs was compared to their respective single drug nanoparticle formulation. While NP[Carf] (IC₅₀=37.4 nM) exhibited an approximate two-fold decrease in IC₅₀ value relative to free carfilzomib (IC₅₀=63.5 nM; FIG. 4A), NP[Dox] and free doxorubicin had very similar IC₅₀ values of 220 nM and 235 nM, respectively (FIG. 4B). Next, the cytotoxicity of NP[Carf+Dox] was evaluated and compared to Carf+Dox as well as NP[Carf]+NP[Dox]. The results demonstrated that NP[Carf+Dox] (IC₅₀=18.7 nM) was more cytotoxic than Carf+Dox (IC₅₀=45 nM) and NP[Carf]+NP[Dox] (IC₅₀=23.1 nM; FIG. 4C).

The reduced efficacy of NP[Carf]+NP[Dox] relative to NP[Carf+Dox] can be attributed to the differing rate at which NP[Dox] is taken up by the cells relative to NP[Carf]. The hydrophobic patches created by doxorubicin on the surface of NP[Dox] which could increase its non-specific cellular interactions and facilitate endocytosis. This would change the therapeutic molar ratio within the cells as doxorubicin would be preferentially taken up mitigating the observed synergy. However, the optimal synergistic ratio is delivered to the cells with NP[Carf+Dox] as both drugs would be taken up at the same rate. Synergistic analysis of the cytotoxicity data for the both NP[Carf+Dox] and NP[Carf]+NP[Dox], based upon the activity of NP[Carf] and NP[Dox], showed that NP[Carf+Dox] (CI=0.584) exhibited more synergy than NP[Carf]+NP[Dox] (CI=0.721). Furthermore, NP[Carf+Dox] had a lower CI value than Carf+Dox (CI=0.898), indicating that NP[Carf+Dox] has improved synergy over the other formulations.

To validate these results, flow cytometric analysis of the early apoptosis marker, annexin V, was performed. NCI-H929 cells were incubated with different free and liposomal formulations of carfilzomib and doxorubicin at a concentration of 12.5 nM for each drug (FIG. 4D). The results demonstrated that the liposomal formulations elicited higher expression levels of annexin V than the free drug formulations. NP[Carf]+NP[Dox] had a minimal increase in annexin V expression compared to NP[Carf]. However, NP[Carf+Dox] had significantly higher expression levels than NP[Carf] and NP[Carf]+NP[Dox], demonstrating the advantages that can be gained by using this formulation. Taken together, NP[Carf+Dox] demonstrated improved efficacy relative to the other combinatorial formulations.

### Example 5

### Determination of the Maximum Tolerated Dose for the Free Carfilzomib and Free Doxorubicin Combination Treatment In Vivo

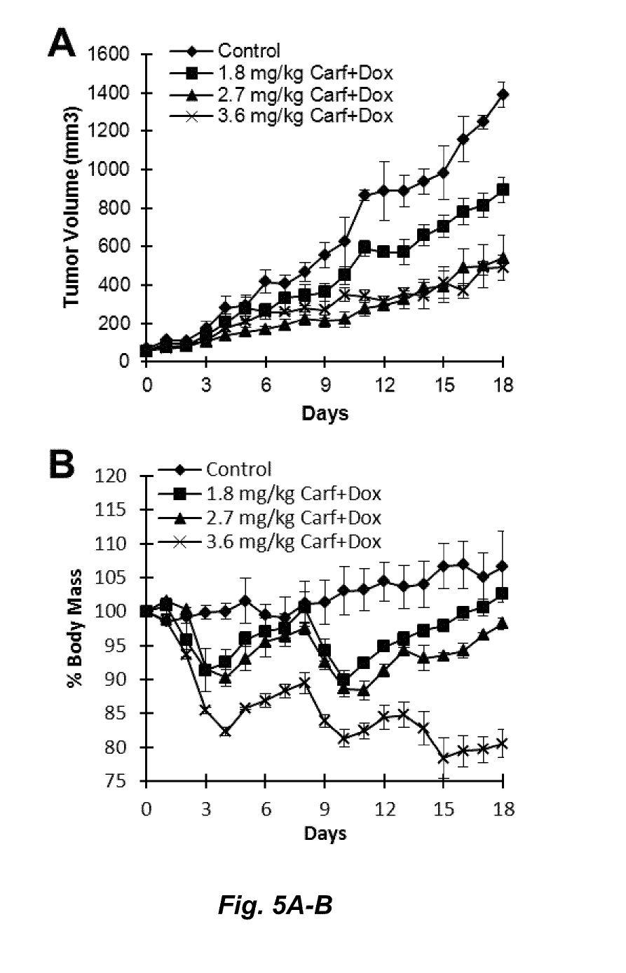

To test if NP[Carf+Dox] is more efficacious than the other formulations, it is imperative to ultimately evaluate them in vivo. In order to do this, we first determined the maximum tolerated dose (MTD; <15% body mass loss) of free carfilzomib and doxorubicin combination treatment at the identified optimal synergistic molar ratio of 1:1. While this will not accurately represent MTD for NP[Carf+Dox], it can still be used to set the dosing parameters for comparison studies since we expect the nanoparticle formulations to be less toxic relative to the free drug formulations. SCID mice were subcutaneously injected with NCI-H929 cells. When the tumors reached a volume of 50 mm³, the mice were randomized into treatment groups and received one of the regimens: i) Control (PBS), ii) 1 mg/kg Carf+0.8 mg/kg Dox combination (1.8 mg/kg Carf+Dox), iii) 1.5 mg/kg Carf+1.2 mg/kg Dox (2.7 mg/kg Carf+Dox), or iv) 2 mg/kg Carf+1.6 mg/kg Dox (3.6 mg/kg Carf+Dox). Treatments were given intravenously on days 1, 2, 8, and 9, modeling the clinical dosing schedule for carfilzomib. Tumor growth and body mass were monitored throughout the study as a measure of therapeutic efficacy and systemic toxicity, respectively. Results demonstrated that mice in the 2.7 mg/kg and 3.6 mg/kg Carf+Dox groups demonstrated significant tumor growth inhibition relative to those that received 1.8 mg/kg (FIG. 5A). While the 2.7 mg/kg and 3.6 mg/kg doses demonstrated similar responses in tumor growth, they differed significantly in systemic toxicity based on average body mass assessment (FIG. 5B). The mice that received 2.7 mg/kg lost, at most, ˜10% body mass and were able to recover most of it by the end of the study. In contrast, mice that received 3.6 mg/kg lost substantially more mass (˜20%) throughout the study and were not able to recover it. Thus, the MTD for Carf+Dox was determined to be 2.7 mg/kg.

### Example 6

### In Vivo Efficacy of the Dual Drug Loaded Liposomes

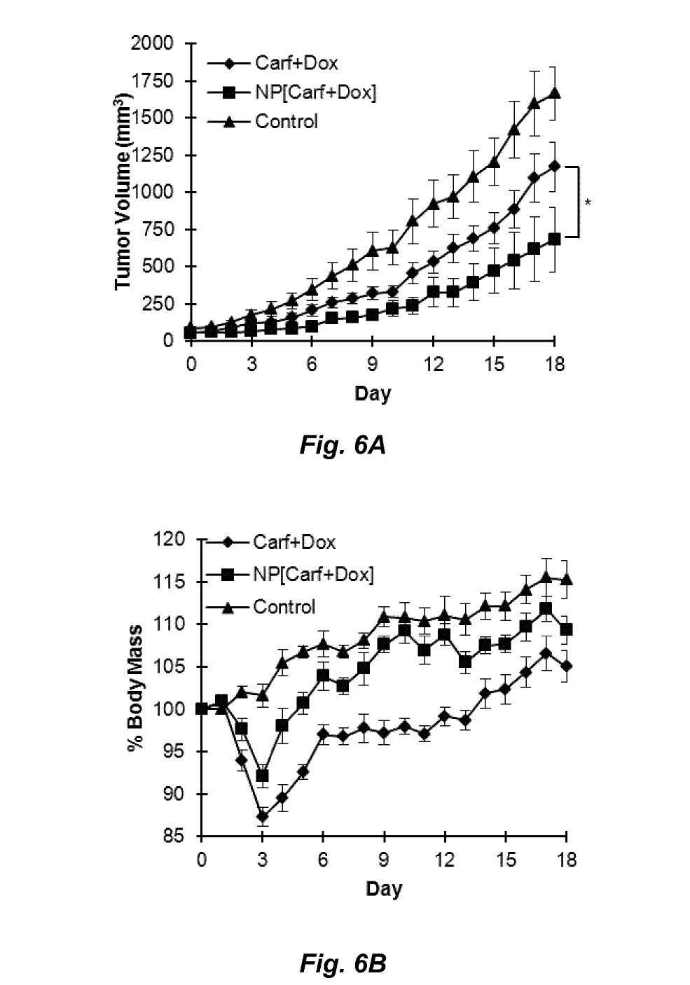

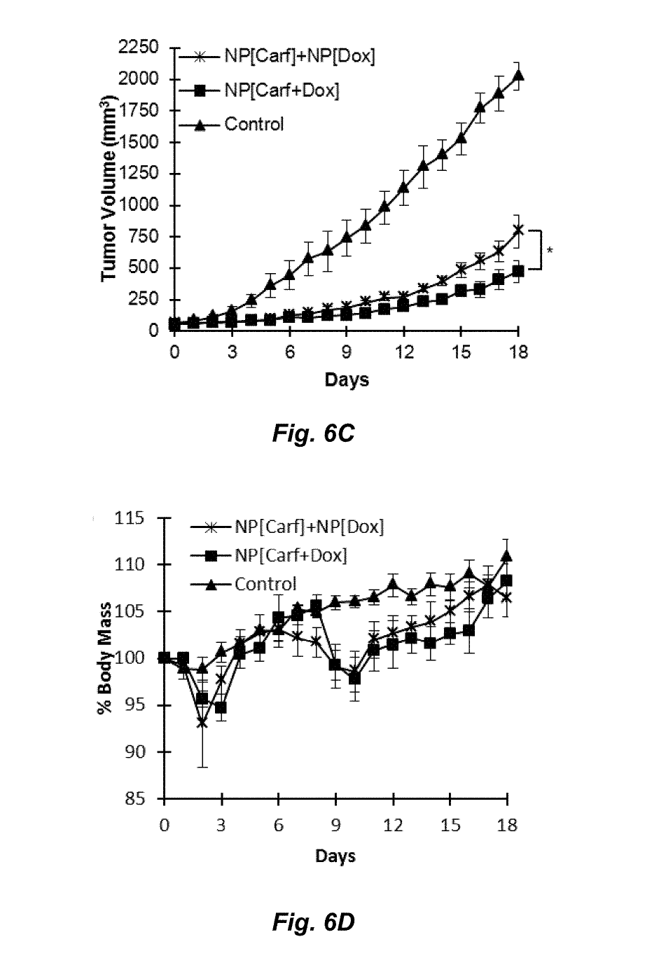

To evaluate the therapeutic potential of NP[Carf+Dox], subcutaneous NCI-H929 tumor bearing SCID mice were randomized into treatment groups when tumors reached a volume of 50 mm³ and were intravenously injected with PBS (Control), Carf+Dox, or NP[Carf+Dox] at a dose of 1 mg/kg carfilzomib+0.8 mg/kg doxorubicin equivalents (1.8 mg/kg total drug) on days 1, 2, 8, and 9. Our results demonstrated that NP[Carf+Dox] significantly inhibited tumor growth inhibition (FIG. 6A) and reduced systemic toxicity relative to Carf+Dox (FIG. 6B). We anticipate that the increased efficacy of NP[Carf+Dox] over Carf+Dox was due the delivery of the drugs at their optimal ratio as well as the other advantages gained from nanoparticle incorporation.

To determine that the efficacy of NP[Carf+Dox] is attributed to the delivery of the therapeutics at their optimal synergistic ratio via single nanoparticle incorporation and not simply the result of the enhanced drug delivery properties gained by nanoparticles, the therapeutic efficacy of NP[Carf+Dox] was compared to NP[Carf]+NP[Dox]. Mice were injected with PBS (Control), NP[Carf+Dox], or NP[Carf]+NP[Dox] to evaluate tumor growth inhibition and systemic toxicity. While both formulations inhibited tumor growth, NP[Carf+Dox] demonstrated greater tumor growth inhibition than NP[Carf]+NP[Dox] (FIG. 6C) while maintaining a similar systemic toxicity profile (FIG. 6D). As expected, both nanoparticle treatment regimens showed minimal weight loss, demonstrating their ability to reduce the overall systemic toxicities associated with the free drugs. However, NP[Carf+Dox] demonstrated significantly greater tumor growth inhibition relative to NP[Carf]+NP[Dox], which indicates that NP[Carf+Dox] was able to deliver both therapeutics to the tumor at their synergistic ratio for an improved effect. The reduced efficacy of NP[Carf]+NP[Dox] could be attributed to sub-optimal drug ratios at the tumor as a result of the differing circulation clearance rates between NP[Carf] and NP[Dox]. Specifically, the surface conjugated doxorubicin in NP[Dox] may facilitate opsonization and increase its clearance rate relative to NP[Carf]. While surface conjugated doxorubicin is also present in NP[Carf+Dox], and still may increase the nanoparticle clearance rate, this affects both therapeutics equally which does not impact the drug ratio delivered to the tumor. Taken together, these results further validate the potential impact that the NP[Carf+Dox] may have in the clinic.

### Example 7

### Pharmaceutical Dosage Forms

The following formulations illustrate representative pharmaceutical dosage forms that may be used for the therapeutic administration of a liposomal composition described herein, or a liposomal composition specifically disclosed herein, (hereinafter ‘Composition X’):

| (i) Injection 1 (1 mg/mL) | mg/mL | |
|---|---|---|
| ‘Composition X’ | 1.0 | |
| Dibasic sodium phosphate | 12.0 | |
| Monobasic sodium phosphate | 0.7 | |
| Sodium chloride | 4.5 | |
| 1.0N Sodium hydroxide solution | q.s. | |
| (pH adjustment to 7.0-7.5) | | |
| Water for injection | q.s. ad 1 | mL |

| (ii) Injection 2 (10 mg/mL) | mg/mL | |
|---|---|---|
| ‘Composition X’ | 10.0 | |
| Monobasic sodium phosphate | 0.3 | |
| Dibasic sodium phosphate | 1.1 | |
| Polyethylene glycol 400 | 200.0 | |
| 0.1N Sodium hydroxide solution | q.s. | |
| (pH adjustment to 7.0-7.5) | | |
| Water for injection | q.s. ad 1 | mL |

| (iii) Aerosol | mg/can |
|---|---|
| ‘Composition X’ | 20 |
| Oleic acid | 10 |
| Trichloromonofluoromethane | 5,000 |
| Dichlorodifluoromethane | 10,000 |
| Dichlorotetrafluoroethane | 5,000 |

These formulations may be prepared by conventional procedures well known in the pharmaceutical art. It will be appreciated that the above pharmaceutical compositions may be varied according to well-known pharmaceutical techniques to accommodate differing amounts and types of active ingredient ‘Composition X’. Aerosol formulation (iii) may be used in conjunction with a standard, metered dose aerosol dispenser. Additionally, the specific ingredients and proportions are for illustrative purposes. Ingredients may be exchanged for suitable equivalents and proportions may be varied, according to the desired properties of the dosage form of interest.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

1. A liposome comprising:
a) a phospholipid;
b) a pegylated lipid;
c) an aqueous core; and
d) at least two different drug components, wherein the drug components comprise a covalently-linked drug-conjugated lipid, an encapsulated drug, or a combination thereof, and wherein the drug components are anticancer drugs;
wherein the diameter of the liposome is about 5 nm to about 200 nm.

2. The liposome of claim 1 wherein the liposome comprises two different drug components, wherein the two different drug components are i) a proteasome inhibitor and an anthracycline, ii) a proteasome inhibitor and a histone deacetylase inhibitor (HDAC inhibitor), or iii) an anthracycline and an HDAC inhibitor.

3. The liposome of claim 2 wherein the two different drug components are present in a synergistic ratio.

4. The liposome of claim 3 wherein the proteasome inhibitor is carfilzomib or bortezomib.

5. The liposome of claim 3 wherein the anthracycline is doxorubicin, amrubicin, daunorubicin, epirubicin, idarubicin, nemorubicin, pixantrone, sabarubicin, or valrubicin.

6. The liposome of claim 3 wherein the HDAC inhibitor is rocilinostat (ACY-1215) or vorinostat.

7. The liposome of claim 3 wherein a drug-conjugated lipid is present and is selected from the group consisting of a bortezomib-prodrug, a histone deacetylase inhibitor-prodrug, and a doxorubicin-prodrug.

8. The liposome of claim 7 wherein the phospholipid comprises 1,2-distearoyl-sn-glycero-3-phosphocholine (DSPC), 1,2-distearoyl-sn-glycero-3-phosphatidylserine (DSPS), 1,2-distearoyl-sn-glycero-3-phosphothethanolamine (DSPE), or hydrogenated soy L-a-phosphatidylcholine (HSPC).

9. The liposome of claim 7 wherein the pegylated lipid comprises DSPE-PEG2000.

10. The liposome of claim 7 wherein the liposome comprises at least one encapsulated drug component and the encapsulated drug component is localized to the aqueous core.

11. The liposome of claim 10 wherein the at least one encapsulated drug component is doxorubicin.

12. The liposome of claim 1 wherein the drug component comprises carfilzomib and a doxorubicin-prodrug, present in a ratio of about 1:1 to about 2:1.

13. The liposome of claim 1 wherein the drug component comprises carfilzomib and doxorubicin, present in a ratio of about 1:1 to about 2:1.

14. The liposome of claim 1 wherein the drug component comprises carfilzomib and rocilinostat, present in a ratio of about 1:1 to about 1:25.

15. The liposome of claim 1 wherein the liposome has a diameter of about 5 nm to about 30 nm, or about 30 nm to about 150 nm.

16. A dual-drug loaded liposome comprising:
a) about 85 wt. % to about 95 wt. % 1,2-distearoyl-sn-glycero-3-phosphocholine (DSPC);
b) about 2 wt. % to about 8 wt. % DSPE-PEG2000;
c) an aqueous core comprising 0.5 wt. % to about 3 wt. % of a first drug component; and
d) 0.5 wt. % to about 3 wt. % of a second drug component, wherein the second drug component is different from the first drug component, the first and second drug components are i) a proteasome inhibitor and an anthracycline, ii) a proteasome inhibitor and a histone deacetylase inhibitor (HDAC inhibitor), or iii) an anthracycline and an HDAC inhibitor,
wherein the diameter of the liposome is about 100 nm to about 130 nm.

17. The liposome of claim 16 wherein the proteasome inhibitor is carfilzomib and the anthracycline is doxorubicin conjugated to 1,2-dipalmitoyl-sn-glycero-3-phosphoethanolamine (DPPE) through a hydrazone linkage, and the lipid bilayer of the liposome comprises the DPPE moiety of the doxorubicin conjugated to DPPE.

18. A method to kill or inhibit the growth of cancer cells comprising contacting the cells with an effective amount of liposomes of claim 1.

19. The method of claim 18 wherein the cancer cells are multiple myeloma cells.

20. A method to treat a multiple myeloma in a subject comprising administering to a subject afflicted with a multiple myeloma an effective amount of the liposome of claim 1.

21. The method of claim 20 wherein the multiple myeloma is refractory multiple myeloma or relapse multiple myeloma.

22. The method of claim 20 wherein the subject is a human subject or mammalian subject.

23. (canceled)

24. (canceled)
